(19) United States
(12) Patent Application Publication
(10) Pub. No.: US 2024/0173524 A1
(43) Pub. Date: May 30, 2024

(54) GUIDEWIRE FEEDER

(71) Applicant: Edwards Lifesciences Innovation (Israel) Ltd., Caesarea (IL)

(72) Inventors: Tal Hammer, Ramat Gan (IL); Meir KUTZIK, Holon (IL); Tal REICH, Moledet (IL); Alexei KOIFMAN, Melbourne (AU)

(21) Appl. No.: 18/059,349

(22) Filed: Nov. 28, 2022

Related U.S. Application Data
(63) Continuation of application No. 16/551,607, filed on Aug. 26, 2019, now Pat. No. 11,534,583.
Application No. 16/551,607 is a continuation of application No. 14/209,171, filed on Mar. 13, 2014, now Pat. No. 10,449,333.
(60) Provisional application No. 61/782,121, filed on Mar. 14, 2013.

(51) Int. Cl. A61M 25/09

(52) U.S. Cl. CPC A61M 25/09041; A61M 2025/09116

(57) ABSTRACT

A guidewire is slidable through a percutaneous medical device. A distal end of the percutaneous medical device is moved in a proximal direction away from an anatomical site of a body of a subject. Simultaneously, a position of a distal end of the guidewire, with respect to the anatomical site, is maintained by moving a housing toward another housing that is coupled to the medical device. Other embodiments are also described.

### CROSS-REFERENCES TO RELATED APPLICATIONS

The present application is a Continuation of U.S. patent application Ser. No. 16/551,607 to Hammer et al., filed Aug. 26, 2019, and titled “Guidewire Feeder,” which is a continuation of U.S. patent application Ser. No. 14/209,171 to Hammer et al., filed Mar. 13, 2014, and titled “Guidewire Feeder,” which claims priority from U.S. provisional patent application 61/782,121 to Hammer et al., filed Mar. 14, 2013, and titled “Guidewire Feeder,” each of which is incorporated herein by reference.

### FIELD OF THE INVENTION

Some applications of the invention relate to medical apparatus and techniques. Some applications of the invention relate more specifically to apparatus and techniques for percutaneous medical procedures, such as those involving the use of a guidewire.

### BACKGROUND

Percutaneous (e.g., transluminal) medical procedures often require the use of a guidewire to facilitate positioning of percutaneous medical devices (e.g., tools, catheters, implants, etc.). Manipulation of the guidewire, such as feeding the guidewire distally into the body of a subject, is often performed by hand.

### SUMMARY OF THE INVENTION

A guidewire feeder is provided, comprising a first housing that defines a first channel therethrough, a second housing that defines a second channel therethrough, and a tubular member that is coupled to the second housing and slidably coupled to the first housing. A guidewire disposed within the first channel, the second channel and the tubular member is moved distally through the feeder by repeatedly moving the second housing toward and away from the first housing such that the tubular member slides into and out of the second housing. Each time the second housing is moved toward the first housing, a portion of the guidewire that is disposed within the tubular member is slid distally, with the tubular member, into the first housing. Each time the second housing is moved away from the first housing, the tubular member slides proximally out of the first housing and over the portion of the guidewire, the guidewire remaining stationary with respect to the first housing. Oscillation of the second housing toward and away from the first housing thereby advances the guidewire through the first channel.

A guidewire-engaging element, coupled to the second housing, is configured to facilitate one-way movement of the guidewire through the second channel, and thereby to facilitate the function of the guidewire feeder described hereinabove. For some applications, an engagement switch is configured to move the guidewire-engaging element between an engaged state in which the guidewire feeder functions as described hereinabove, and a disengaged state in which the guidewire is slidable through the second channel in either direction (e.g., so as to facilitate initial threading of the guidewire through the guidewire feeder).

For some applications, the guidewire feeder is configured to be coupled to a medical device for percutaneous procedures. For some applications the guidewire feeder is used to advance the guidewire distally through the medical device. For some applications, the guidewire feeder is used to facilitate withdrawal of the medical device over the guidewire, while maintaining the position of the guidewire (e.g., a distal end thereof) with respect to the body of the subject.

There is therefore provided, in accordance with an application of the present invention, apparatus for use with a guidewire, the apparatus including:
a first housing, shaped to define a first channel through which the guidewire is slidable;
a second housing, shaped to define a second channel through which the guidewire is slidable in at least a first direction toward the first housing;
a guidewire-engaging element, disposed within the second housing, and configured to inhibit the guidewire from sliding through the second channel in a second direction away from the first housing; and
a tubular member:
   shaped to define a lumen therethrough, through which the guidewire is slidable, and
   coupled to the second housing and slidably coupled to the first housing.

In an application, the tubular member is slidable within the first channel.

In an application:
the guidewire-engaging element has an engaged state and a disengaged state, and is configured to inhibit the guidewire from sliding through the second channel in the second direction, only when in the engaged state, and
the apparatus further includes an engagement switch, configured to reversibly move the guidewire-engaging element between the engaged state and the disengaged state.

In an application, the apparatus is configured to facilitate percutaneous advancement of the guidewire by a physician, while the physician (a) directly holds the second housing of the device with only one finger and one thumb, and (b) does not directly hold the first housing.

In an application, the lumen of the tubular member provides communication between the first channel and the second channel.

In an application, the tubular member is more rigid than the guidewire, and is configured to inhibit bending of a portion of the guidewire that is disposed within the lumen of the tubular member.

In an application, the first channel, the second channel, and the lumen are collinear.

In an application, the first channel, the second channel, and the lumen define an axis, and the second housing is rotatable around the axis.

In an application, the tubular member is coupled to the second housing and slidably coupled to the first housing such that the second housing is slidable toward the first housing and away from the first housing.

In an application, the apparatus is configured such that, while a portion of the guidewire is disposed within the second channel, when the second housing is moved toward the first housing, the guidewire moves in the first direction.

In an application, the apparatus is configured such that, while the portion of the guidewire is disposed within the second channel, oscillation of the second housing between the first direction and the second direction moves the guidewire in the first direction.

In an application, the apparatus is configured such that, when the second housing slides toward the first housing, the tubular member slides into the first housing.

In an application, the apparatus is configured such that, while the portion of the guidewire is disposed within the second channel, and a portion of the guidewire is disposed within the lumen of the tubular member, when the second housing is moved toward the first housing, the portion of the guidewire that is disposed within the lumen of the tubular member moves while within the tubular member into the first housing, without moving with respect to the tubular member.

In an application, the apparatus is configured such that, when the second housing slides toward the first housing, the tubular member slides into the first channel.

In an application, the apparatus is further for use with a device for facilitating percutaneous procedures, and the first housing is configured to be coupled to the device.

In an application, the first housing includes a coupling member, configured to facilitate coupling the first housing to the device.

In an application, the coupling member includes a generic coupling member, configured to facilitate coupling of the first housing to any of a range of devices for facilitating percutaneous procedures.

In an application, the coupling member includes a strap.

In an application, the guidewire-engaging element is configured such that, while the guidewire is disposed within the second channel, when the guidewire moves in the second direction, the guidewire-engaging element responsively grips the guidewire.

In an application, the guidewire-engaging element is pivotally coupled to the second housing.

In an application, the guidewire-engaging element defines a guidewire-engaging edge, and is configured such that, while the guidewire is disposed within the second channel, the guidewire-engaging edge is disposed against the guidewire at a nonzero angle, such that movement of the guidewire in the second direction through the second channel causes the guidewire-engaging edge to press into the guidewire.

In an application, the guidewire-engaging element is configured such that, while the guidewire is disposed within the second channel, when the guidewire-engaging edge presses into the guidewire, the nonzero angle becomes closer to a right angle.

In an application, the guidewire-engaging element is configured such that, while the guidewire is disposed within the second channel, the guidewire-engaging edge is disposed against the guidewire at a nonzero angle that is not a right angle.

In an application, the guidewire-engaging edge is configured such that, while the guidewire is disposed within the second channel, the guidewire-engaging edge is disposed against the guidewire at an obtuse angle with respect to a portion of the guidewire that is disposed closer to the tubular member than is the guidewire-engaging edge.

There is further provided, in accordance with an application of the present invention, a method for use with a guidewire, the method including:
providing apparatus including:
   a first housing,
   a second housing, and
   a tubular member, coupled to the second housing and slidably coupled to the first housing;
sliding a portion of the guidewire that is disposed within the tubular member in a first direction through the first housing by sliding the tubular member into the first housing by moving the second housing toward the first housing; and
moving the second housing away from the first housing without sliding the guidewire in a second direction through the first housing.

In an application, the method further includes percutaneously advancing the guidewire by sliding the portion of the guidewire in the first direction.

In an application, moving the second housing toward the first housing includes moving the second housing toward the first housing using only one finger and one thumb.

In an application, sliding the tubular member into the first housing includes sliding, into the first housing, a tubular member that is more rigid than the guidewire and is configured to inhibit bending of the portion of the guidewire.

In an application, the method further includes coupling the first housing to a device for facilitating percutaneous procedures.

In an application, the method further includes coupling the first housing to a body of a subject.

In an application, the apparatus includes a guidewire-engaging element, disposed within the second housing, and having an engaged state and a disengaged state, and the method further includes:
feeding the guidewire into at least the second housing while the guidewire-engaging element is in the disengaged state; and
subsequently moving the guidewire-engaging element into the engaged state.

In an application, moving the guidewire-engaging element into the engaged state includes moving the guidewire-engaging element into the engaged state using an engagement switch, configured to reversibly move the guidewire-engaging element between the engaged state and the disengaged state.

In an application, the apparatus includes a guidewire-engaging element coupled to the second housing, and moving the second housing toward the first housing includes moving the second housing toward the first housing such that the guidewire-engaging element responsively grips the guidewire.

In an application, moving the second housing toward the first housing includes moving the second housing toward the first housing such that the guidewire-engaging element responsively pivots with respect to the second housing.

In an application, the guidewire-engaging element defines a guidewire-engaging edge, and moving the second housing toward the first housing includes moving the second housing toward the first housing such that the guidewire-engaging edge presses into the guidewire.

There is further provided, in accordance with an application of the present invention, a method, including:
moving a distal end of a percutaneous medical device in a proximal direction away from an anatomical site of a body of a subject; and
simultaneously, maintaining a position, with respect to the anatomical site, of a distal end of a guidewire that is slidable through the medical device by moving, toward a first housing that is coupled to the medical device, a second housing.

In an application, moving the distal end of the medical device includes pulling a handle of the medical device in the proximal direction using a first hand, and moving the second housing toward the first housing includes moving the second housing toward the first housing using a second hand.

In an application, moving the distal end of the medical device includes moving the distal end of the medical device at a first speed, and moving the second housing toward the first housing includes moving the second housing toward the first housing at a second speed that is relative to the first speed.

In an application, moving the distal end of the medical device includes moving the distal end of the medical device a first distance, and moving the second housing toward the first housing includes moving the second housing toward the first housing a second distance that is relative to the first distance.

In an application, moving the second housing toward the first housing includes moving the second housing toward the first housing such that a guidewire-engaging element, coupled to the second housing, responsively grips the guidewire.

In an application, moving the second housing toward the first housing includes moving the second housing toward the first housing such that the guidewire-engaging element responsively pivots with respect to the second housing.

In an application, the guidewire-engaging element defines a guidewire-engaging edge, and moving the second housing toward the first housing includes moving the second housing toward the first housing such that the guidewire-engaging edge presses into the guidewire.

In an application, the method further includes sliding at least the second housing over a proximal end of the guidewire and distally along the guidewire, and coupling at least the second housing to the medical device.

In an application, sliding at least the second housing distally along the guidewire includes sliding at least the second housing distally along the guidewire while a guidewire-engaging element, coupled to the second housing, is in a disengaged state thereof, and the method further includes, subsequently to coupling at least the second housing to the medical device and prior to maintaining the position of the distal end of the guidewire, moving the guidewire-engaging element into an engaged state thereof.

In an application, moving the guidewire-engaging element into the engaged state includes moving the guidewire-engaging element into the engaged state using an engagement switch, configured to reversibly move the guidewire-engaging element between the engaged state and the disengaged state.

In an application, moving the second housing toward the first housing includes moving the second housing at least 1 cm toward the first housing.

In an application, moving the second housing toward the first housing includes moving the second housing at least 3 cm toward the first housing.

In an application, moving the second housing toward the first housing includes moving the second housing at least 4 cm toward the first housing.

In an application, moving the second housing toward the first housing includes sliding into the first housing, a tubular member that is coupled to the second housing and slidably coupled to the first housing.

In an application, sliding the tubular member into the first housing includes sliding a portion of the guidewire that is disposed within the tubular member into the first housing without the portion of the guidewire moving with respect to the tubular member.

In an application, sliding the tubular member into the first housing includes sliding, into the first housing, a tubular member that is more rigid than the guidewire and is configured to inhibit bending of the portion of the guidewire.

In an application, the method further includes subsequently sliding the tubular member out of the first housing by moving the second housing away from the first housing, without sliding the guidewire with respect to the first housing.

The present invention will be more fully understood from the following detailed description of embodiments thereof, taken together with the drawings, in which:

### BRIEF DESCRIPTION OF THE DRAWINGS

FIGS. 1-2 are schematic illustrations of a guidewire feeder for facilitating handling of a guidewire, in accordance with some applications of the invention;

FIGS. 3A-B and 4A-B are schematic illustrations of a guidewire feeder, in accordance with some applications of the invention;

FIGS. 5A-D are schematic illustrations of a guidewire feeder being used to advance the guidewire, in accordance with some applications of the invention;

FIG. 6 is a schematic illustration of a guidewire feeder being used to advance the guidewire into the femoral vein of a subject, in accordance with some applications of the invention;

FIG. 7 is a schematic illustration of a guidewire feeder, for use with the guidewire and a cannula, in accordance with some applications of the invention; and

FIGS. 8A-B are schematic illustrations of a guidewire feeder being used to facilitate movement of a medical device with respect to at least a portion of a guidewire, in accordance with some applications of the invention.

### DETAILED DESCRIPTION OF EMBODIMENTS

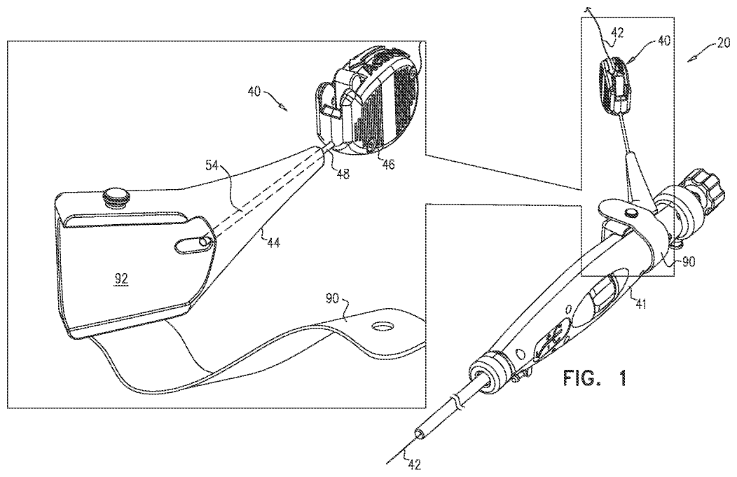

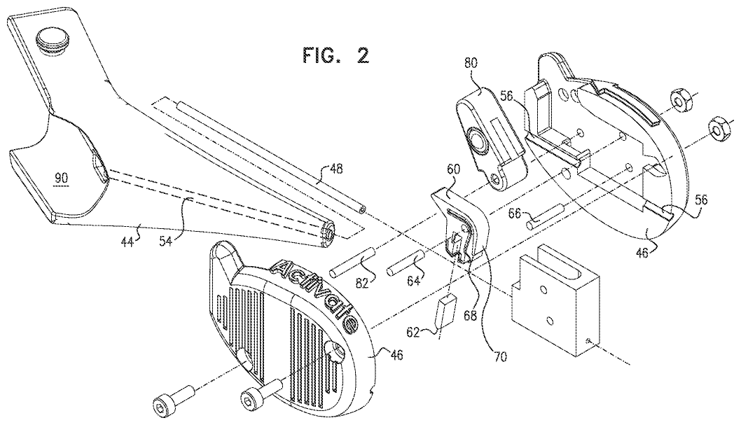

Reference is made to FIGS. 1-2, which are schematic illustrations of a guidewire feeder 40 for facilitating handling of a guidewire 42, in accordance with some applications of the invention. FIG. 1 shows a solid view of guidewire feeder 40 coupled to a medical device 41 for facilitating percutaneous (e.g., transluminal) procedures, and FIG. 2 shows an exploded view of the guidewire feeder. Together, guidewire feeder 40 and guidewire 42 define a system 20. It is to be noted that device 41 is shown as a non-limiting illustrative example of a device to which guidewire feeder 40 is couplable.

Feeder 40 comprises a first housing 44, a second housing 46, and a tubular member 48 that is coupled to the second housing and slidably coupled to the first housing. First housing 44 defines a first channel 54 therethrough, second housing 46 defines a second channel 56 therethrough, and tubular member 48 defines a lumen therethrough. Tubular member 48 (e.g., the lumen thereof) typically provides communication between first channel 54 and second channel 56. First channel 54, tubular member 48, and second channel 56 are typically collinear. Further typically, tubular member 48 is slidably coupled to first housing 44 by being slidable within first channel 54. Tubular member 48 is typically fixedly coupled to second housing, such as by a portion of the tubular member being fixedly coupled within a portion of second channel 56. Feeder 40 further comprises a guidewire-engaging element 60 that is coupled to and/or disposed within second housing 46, and is configured to selectively inhibit the guidewire from sliding through the second channel away from the first housing (e.g., and to not inhibit the guidewire from sliding through the second channel toward the first housing).

Guidewire 42 typically fits snugly within channel 54, channel 56 and/or tubular member 48, but not tightly enough to prevent sliding thereof (e.g., such that the guidewire is subject to friction that is not significantly higher than that to which it is subjected within medical device 41). For some applications, inner surfaces of channel 54, channel 56 and/or tubular member 48 are coated with a low-friction coating so as to further reduce inhibition of advancement of the guidewire. For some applications, guidewire 42 itself is subjected to a friction-reducing treatment (e.g., heat-treated and/or coated with a low-friction coating). Guidewire-engaging element 60 is configured to grip guidewire 42 despite this treatment of the guidewire. Indeed, some prior art guidewires are subjected to a friction-reducing treatment, and feeder 40 may be particularly useful in handling such guidewires.

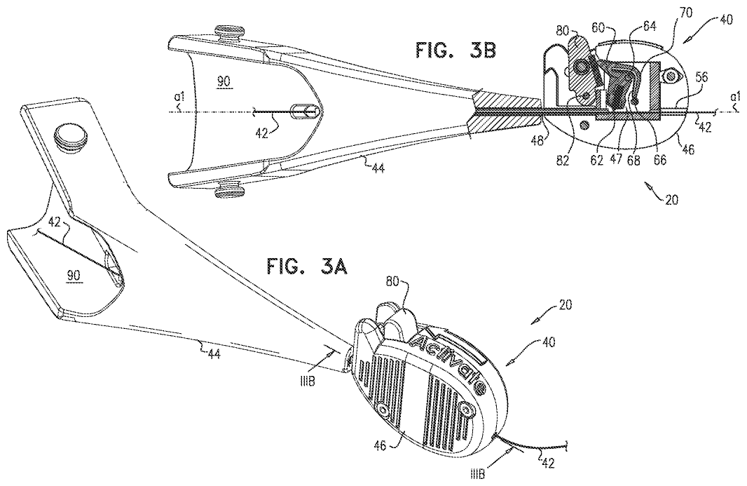

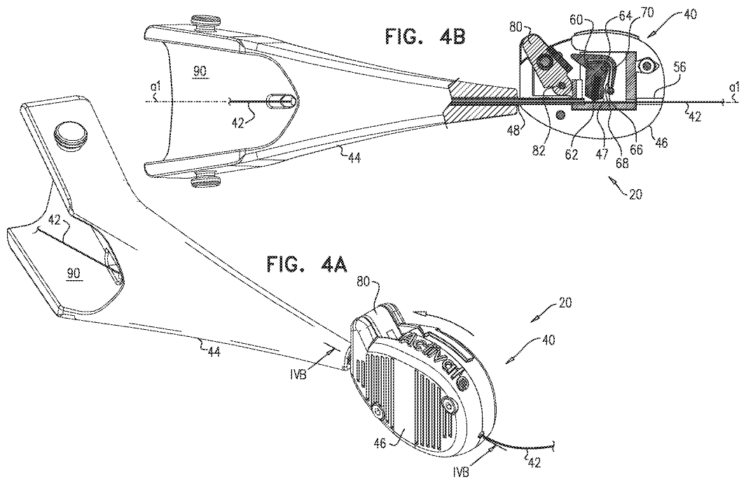

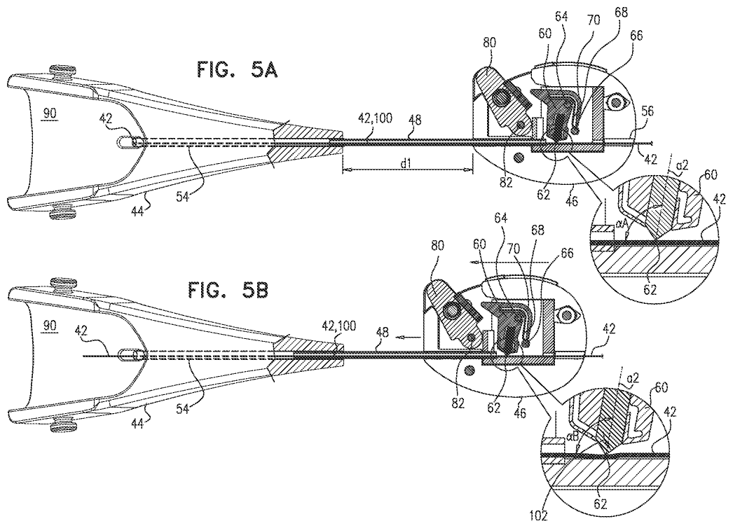

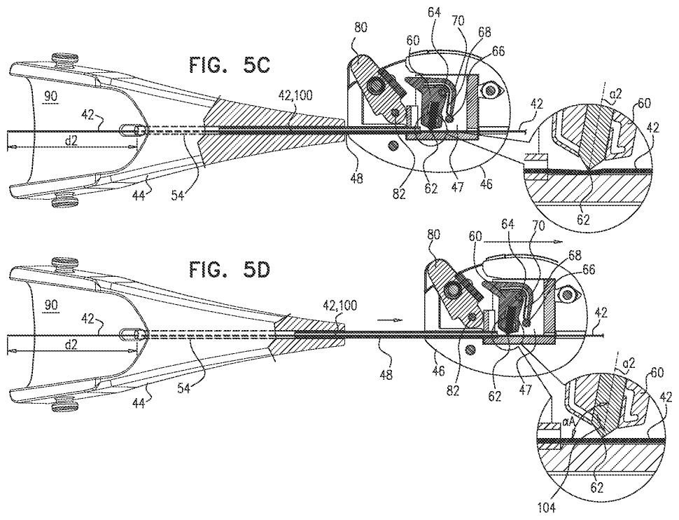

When second housing 46 is moved toward first housing 44, at least a portion of tubular member 48 slides into the first housing. While a portion of guidewire 42 is disposed within feeder 40, when second housing 46 is moved toward first housing 44, a portion of the guidewire that is disposed within tubular member 48 is pushed, along with the tubular member, into the first housing (e.g., into first channel 54). In contrast, when second housing 46 is moved away from first housing 44, although tubular member 48 slides out of the first housing, the guidewire does not follow. Thereby, oscillation of second housing 46 toward and away from first housing 44 (e.g., along a longitudinal axis a1 defined by first channel 54, tubular member 48, and second channel 56; shown in FIGS. 3B and 4B) moves the guidewire in a single direction through the first housing. This is described in more detail with respect to FIGS. 5A-D.

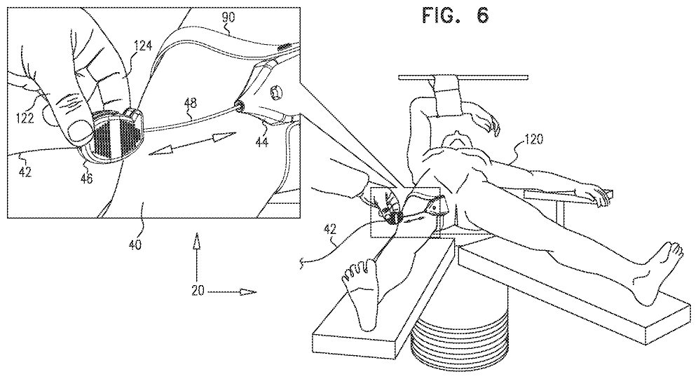

Percutaneous (e.g., transluminal) guidewires are typically flexible and thereby imparting a pushing force on a guidewire results in bending (e.g., kinking or buckling) of a portion of the guidewire that is distal to the point of pushing, e.g., between the point of pushing and a point of resistance, such as an entry point (e.g., to a body of a subject and/or to another medical device). This makes pushing of the guidewire (e.g., distally into the body of the subject) difficult and/or inefficient. In system 20, the point of pushing is at and/or within second housing 46 (e.g., at guidewire-engaging element 60) and, as shown in FIG. 1, first housing 44 is typically reversibly coupled (e.g., reversibly attached) to the other medical device (e.g., medical device 41), such that guidewire 42 is generally not exposed between the first housing and the medical device. Thereby, guidewire feeder 40 provides rigidity (e.g., support) to the aforementioned portion of the guidewire that is disposed between the pushing point and the entry point to the medical device. Furthermore, for applications in which guidewire 42 extends through the medical device, the guidewire is typically generally not exposed between the first housing and a distal portion of the medical device (i.e., a portion of the medical device that is furthest from feeder 40). Still further, guidewire 42 typically fits snugly within second channel 56, tubular member 48, first channel 54, and the medical device (e.g., a lumen defined by the medical device). Thereby, the guidewire, from the first housing to the distal portion of the medical device, is typically inhibited from bending. It is to be noted that throughout this application, including the specification and the claims, such inhibiting of bending refers to undesired bending (e.g., kinking and/or bucking), such as that described earlier in this paragraph as occurring between a point of pushing and a point of resistance, and does not include desired bending such as that caused by confirmation of the guidewire to a particular shape of feeder 40 and/or medical device 41 (e.g., so as to navigate the vasculature of the subject). Guidewire feeder 40 may alternatively be coupled directly to the body of the subject (e.g., as described hereinbelow with reference to FIG. 6).

It is particularly notable that tubular member 48, which is typically more rigid than guidewire 42, provides rigidity to the portion of the guidewire that is disposed between second housing 46 and first housing 44, and thereby inhibits bending of this portion of the guidewire. The rigidity provided by guidewire feeder 40 (e.g., tubular member 48 thereof) typically allows a longer portion of guidewire 42 to be pushed in each ‘stroke’, e.g., compared to pushing the guidewire by hand. For example, each oscillation of feeder 40 may advance the guidewire by more than 1 cm, e.g., more than 3 cm, such as more than 4 cm.

It is to be noted that the term “generally not exposed” does not necessarily preclude small portions of the guidewire from being exposed, such as portions that are less than 1 cm in length; typically less than 3 mm of guidewire (e.g., less than 1 mm of guidewire) is exposed. It is also to be noted that although tubular member 48 is typically more rigid than guidewire 42, the tubular member may be somewhat flexible, such as to accommodate use of feeder 40 at different angles, e.g., due to position of the feeder, the subject, the physician, etc.

To facilitate coupling of guidewire feeder 40 to medical device 41 and/or the body of the subject, the guidewire feeder typically comprises a coupling member 90, such as a strap. Coupling member 90 may alternatively or additionally comprise a pin, a latch, and/or another coupling member. Feeder 40 (e.g., first housing 44 thereof) may additionally comprise an indicator, such as a visual and/or tactile indicator to facilitate positioning of the feeder with respect to medical device 41.

For some applications, coupling member 90 is configured to be coupled to a specific medical device, thereby facilitating coupling of feeder 40 to the specific medical device. For some applications, coupling member 90 is couplable to a range of medical devices; that is, the coupling member comprises a generic coupling member. For example, when coupling member 90 comprises a strap, the strap may be placeable around a portion of a medical device without having being matched to that particular medical device. Typically, first housing 44 is shaped to facilitate coupling to the specific medical device and/or the range of medical devices, such as by being shaped to define a concavity 92 in which a portion of the medical device is placeable. Device 41 typically defines a conduit therethrough, and an opening via which the guidewire is introducible into the conduit. Feeder 40 is typically configured to be coupled to device 41 such that first channel 54 (e.g., an end thereof) is aligned with the opening of the conduit of device 41, such that guidewire 42 can slide freely between the second channel and the conduit.

Reference is now made to FIGS. 3A-B, and 4A-B, which are schematic illustrations of guidewire feeder 40, in accordance with some applications of the invention. Typically, guidewire-engaging element 60 is reversibly engageable (e.g., movable between an engaged state and a disengaged state thereof) using an engagement switch 80. FIGS. 3A-B show solid and cross-sectional views, respectively, of feeder 40 in the disengaged state, and FIGS. 4A-B show solid and cross-sectional views, respectively, of the feeder in the engaged state. Typically, guidewire-engaging element 60 is not in contact with guidewire 42 in the disengaged state, and is in contact with the guidewire in the engaged state.

Engagement switch 80 is typically pivotally coupled, by a bearing 82, to second housing 46, such as with at least part of switch 80 being disposed within a space 47 defined by the second housing. An operating physician may move switch 80 (e.g., using a thumb), so as to move element 60 between (1) the disengaged state, in which the guidewire is typically freely slidable in both directions through second channel 56, e.g., so as to introduce the guidewire through the second channel, and (2) the engaged state, in which the guidewire is inhibited from moving through the second channel away from the first housing, e.g., so as to use feeder 40 to advance the guidewire distally into the body of the subject. Typically, engagement switch 80 applies a force to guidewire-engaging element 60 so as to move the guidewire-engaging element into the disengaged state. For example, a spring 68 may be configured to move element 60 into the engaged state by applying a force, and switch 80 may be configured to (1) move the guidewire-engaging element into the disengaged state by applying an opposing force to the force applied by the spring, and (2) move the guidewire-engaging element into the engaged state by removing the opposing force, thereby allowing the spring to move the guidewire-engaging element into the engaged state.

For some applications, guidewire-engaging element 60 defines a protrusion 70 that is configured to abut an abutment 66 (e.g., defined by second housing 46), so as to limit rotation of the guidewire-engaging element around bearing 64, e.g., to prevent guidewire-engaging element from rotating too far when engagement switch 80 moves the guidewire-engaging element into the disengaged state.

Reference is made to FIGS. 5A-D, which are schematic illustrations of guidewire feeder 40, in the engaged state thereof, being used to advance guidewire 42, in accordance with some applications of the invention. While element 60 is in the engaged state, when the guidewire moves through channel 56 toward first housing 44, element 60 responsively grips the guidewire (e.g., increases its grip on the guidewire compared to when the guidewire moves through channel 56 away from the first housing).

For some applications, and as shown in the figures, guidewire-engaging element 60 defines or comprises a guidewire-engaging edge 62 and is configured such that, while the guidewire is disposed within second channel 56, and element 60 is in the engaged state, the guidewire-engaging edge is disposed against the guidewire at an angle such that if the guidewire begins to move through channel 56 away from first housing 44, element 60 grips the guidewire (e.g., edge 62 presses into and/or catches against the guidewire). When guidewire 42 moves through channel 56 toward first housing 44 (i.e., in the opposite direction), guidewire-engaging element 60 does not grip the guidewire, and the guidewire slides past the guidewire-engaging element, typically with edge 62 in light contact with the guidewire. Typically, edge 62 comprises and/or is defined by a hard material, such as cubic zirconia, aluminium oxide (e.g., alpha-aluminium oxide), cemented carbide (e.g., widia), and/or a hardened metal. For some applications, edge 62 defines a plurality of teeth. For some applications, edge 62 comprises a soft material, such as silicone rubber, that applies friction to the guidewire.

FIG. 5A shows guidewire feeder 40 in a state in which second housing 46 is at a maximum distance d1 from first housing 44 (e.g., a starting position). Typically, when guidewire 42 is stationary within channel 56, guidewire-engaging edge 62 is disposed with respect to guidewire 42 (e.g., against guidewire 42) at a nonzero angle alpha_A that is further typically not a right angle (FIG. 5A). Angle alpha_A is typically an obtuse angle defined between a longitudinal axis a2 of guidewire-engaging edge 62, and a portion of guidewire 42 and/or axis a1 that is disposed closer to tubular member 48 than is the guidewire-engaging edge. An angle defined between guidewire-engaging edge 62 and a portion of channel 56 that is disposed closer to tubular member 48 than is the guidewire-engaging edge, is also typically obtuse.

Guidewire-engaging element 60 is typically pivotally coupled, by a bearing 64, to second housing 46, such as within space 47. Typically, guidewire-engaging element is maintained in the engaged state thereof by spring 68 (e.g., when engagement switch 80 does not hold element 60 in the disengaged state). For example, spring 68 may bias guidewire-engaging element 60 such that edge 62 is disposed against guidewire 42, e.g., by rotating the guidewire-engaging element around bearing 64. That is, spring 68 typically maintains guidewire-engaging element 60 (e.g., guidewire-engaging edge 62 thereof) (1) disposed against guidewire 42, and/or (2) disposed at a predetermined angle with respect to the guidewire.

When second housing 46 is moved toward first housing 44, guidewire 42 begins to move through channel 56, guidewire-engaging element 60 responsively grips the guidewire (e.g., edge 62 presses into and/or catches against the guidewire), and the guidewire is thereby pushed toward the first housing (FIG. 5B). Typically, and as shown by arrow 102 in FIG. 5B, guidewire-engaging element 60 grips the guidewire by rotating slightly, e.g., around bearing 64, such that an angle alpha_B, defined between axis a2 and the portion of guidewire 42 and/or axis a1 that is disposed closer to tubular member 48 than is the guidewire-engaging edge, is smaller than angle alpha_A. Angle alpha_B is typically closer to a right angle than is angle alpha_A. It is to be noted that a portion 100 of guidewire 42 that is disposed within tubular member 48 is moved, while stationary within the tubular member, into first channel 54.

FIG. 5C shows second housing 46 stationary at a closest position thereof to first housing 44. Typically, in this position, second housing 46 abuts first housing 44. Although FIG. 5C shows guidewire-engaging element 60 gripping (e.g., edge 62 pressing into and/or catching against) guidewire 42, for some applications, in this stationary position (and/or in other stationary positions), the guidewire-engaging element does not grip the guidewire, but instead only grips the guidewire when the guidewire moves in the appropriate direction with respect to the guidewire-engaging element. It is to be noted that a length d2 of guidewire 42 that has advanced out of first housing 44 since the state shown in FIG. 5A, is typically approximately equal to distance d1.

FIG. 5D shows second housing 46 being moved away from first housing 44 again, e.g., toward the starting position shown in FIG. 5A. When second housing 46 is moved away from first housing 44, guidewire-engaging element 60 does not grip guidewire 42, and the second housing slides over the guidewire, typically with edge 62 in light contact with the guidewire. That is, guidewire 42 moves through channel 56 in the opposite direction to that shown in FIG. 5B. Guidewire 42 typically remains stationary with respect to first housing 44. Typically, and as shown by arrow 104 in FIG. 5D, guidewire-engaging element 60 typically releases its grip on the guidewire by rotating slightly, e.g., around bearing 64, as guidewire 42 begins to move through channel 56. It will be understood that oscillation of second housing 46 toward and away from first housing 44, results in a net movement of guidewire through guidewire feeder 40 in a direction from second housing 46 to first housing 44.

For some applications, some of the force applied to second housing 46 to move the second housing toward first housing 44, increases gripping of guidewire 42 by guidewire-engaging element 60, and release of this force (e.g., when moving the second housing away from the first housing) releases at least some of this gripping (e.g., such that the gripping is overcome by friction between the guidewire and device 41 and/or the first housing).

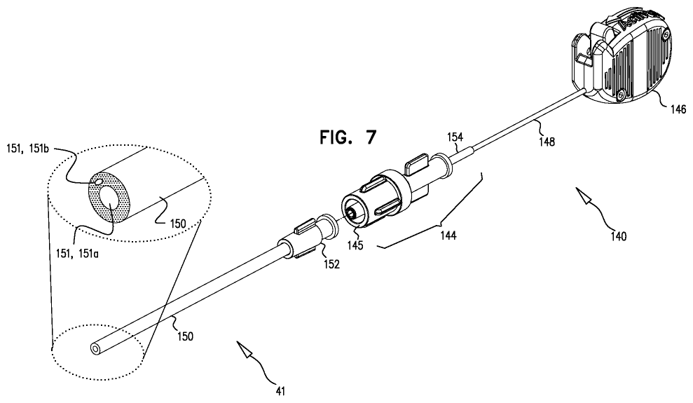

Reference is made to FIG. 6, which is a schematic illustration of guidewire feeder 40 being used to advance guidewire 42 into the femoral vein of a subject 120, in accordance with some applications of the invention. As described hereinabove (e.g., with reference to FIG. 1), for some applications guidewire feeder 40 is configured to be used with another medical device, typically by being coupled to the other medical device (e.g., using coupling member 90). For some applications, and as shown in FIG. 6, guidewire feeder 40 is configured to be coupled to the subject being treated, such as by securing coupling member 90 around the thigh of the subject. For some such applications, first housing 44 is shaped to define, or is coupled to, a cannula that facilitates entry of guidewire 42 into a blood vessel of the subject, such as the femoral vein of the subject (e.g., as described with reference to FIG. 7, mutatis mutandis). It is to be noted that guidewire feeder 140, described with reference to FIG. 7, may be used with the techniques described with reference to FIG. 6, mutatis mutandis.

Reference is made to FIG. 7, which is a schematic illustration of a guidewire feeder 140, for use with guidewire 42 and a cannula 150 that defines at least a first lumen 151 therethrough, in accordance with some applications of the invention. Together, guidewire feeder 140 and cannula 150 define a system 160. System 160 typically further comprises guidewire 42, described hereinabove. For some applications, cannula 150 defines a secondary lumen 153 in the wall of the catheter, lumen 153 typically providing dedicated access for guidewire 42, and lumen 151 typically providing access for other apparatus. Typically, when a dedicated guidewire lumen such as lumen 153 is provided, the guidewire fits snugly within that lumen. It is hypothesized that this snug fit facilitates the pushing of the guidewire without the guidewire bending, and/or makes the use of guidewire feeder 40 more advantageous due to increased friction on the guidewire.

For some applications guidewire feeder 140 comprises the same components of and/or performs the same functions of guidewire feeder 40, described hereinabove, mutatis mutandis. For example, guidewire feeder 140 comprises a first housing 144, a second housing 146, and a tubular member 148 that is coupled to the second housing and slidably coupled to the first housing. First housing 144 defines or is coupled to a pipe 154 that defines a first channel therethrough, which is typically analogous to first channel 54 of guidewire feeder 40, described hereinabove. Second housing 146 defines a second channel therethrough (not shown), and tubular member 148 defines a lumen therethrough, and provides communication between the first channel and the second channel. Typically, tubular member 148 is slidably coupled to first housing 144 by being slidable within pipe 154 (e.g., within the first channel). Tubular member 148 is typically fixedly coupled to second housing, such as by a portion of the tubular member being fixedly coupled within a portion of the second channel. Guidewire feeder 140 further comprises a guidewire-engaging element (not shown) that is coupled to and/or disposed within second housing 146, and is configured to selectively inhibit the guidewire from sliding through the second channel away from the first housing.

Typically, the guidewire-engaging element of guidewire feeder 140 comprises guidewire-engaging element 60, described hereinabove. As described hereinabove for guidewire feeder 40, oscillation of the second housing toward and away from the first housing moves the guidewire disposed therewithin in a single direction through the first housing. First housing 144 is configured to be reversibly coupled (e.g., reversibly attached) to cannula 150, so as to facilitate advancement of guidewire 42 into the body of the subject, e.g., as described for the coupling of guidewire feeder 40 to medical device 41 hereinabove, mutatis mutandis. For some applications, cannula 150 comprises medical device 41. Cannula 150 typically comprises a coupling portion 152, configured to be reversibly coupled to first housing 144, which is typically shaped to define a complementary coupling portion 145. For some applications, coupling portion 152 comprises a valve or similar element to inhibit blood from flowing out via the coupling portion. For some applications, coupling portions 152 and 145 comprise Luer-type fittings. For some applications, guidewire-feeder 140 is configured to be coupled to a generic cannula (e.g., cannula 150 defines a generic cannula). For some applications, guidewire feeder 140 is identical to guidewire feeder 40, except for the shape and/or coupling properties of first housing 144. It is to be noted that in this context, throughout this application, including the specification and the claims, the term “cannula” is used as a general term for a percutaneous access device, and may include, by way of example and not limitation, a hollow needle or a catheter.

Typically, guidewire 42 is typically generally not exposed between second housing 146 and a distal end of cannula 150 (i.e., the end of the cannula that is furthest from feeder 140). For example, cannula 150 may be transluminally advanced to the heart of the subject, and guidewire 42 is enclosed within feeder 140 and cannula 150, from when the guidewire enters second housing 146 until it emerges from the distal end of the cannula in the heart of the subject. That is, the guidewire is inhibited from bending from a point of pushing of the guidewire (i.e., second housing 146, e.g., the guidewire-engaging element thereof), to the distal end of the cannula.

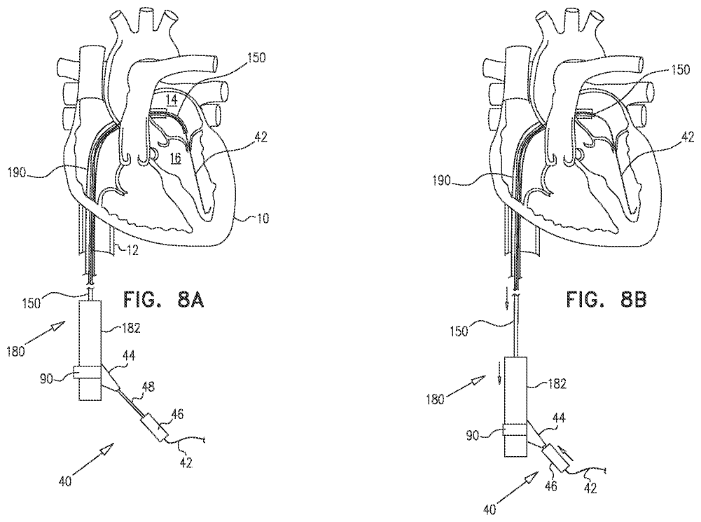

Reference is made to FIGS. 8A-B, which are schematic illustrations of guidewire feeder 40 being used to facilitate movement of a medical device 180 with respect to at least a portion of guidewire 42, in accordance with some applications of the invention. For some applications, medical device 180 comprises medical device 41, described hereinabove. For some applications, medical device 180 comprises a handle 182 and a cannula, such as cannula 150, described with reference to FIG. 7, mutatis mutandis. Guidewire feeder 40 is couplable to device 180 such that first housing 44 (e.g., first channel 54 thereof) is aligned with an entry point on device 180 for guidewire 42. Typically, guidewire feeder 40 is couplable to handle 182 using coupling member 90, handle 182 defining the entry point for guidewire 42 into device 180.

FIG. 8A shows medical device 180 with cannula 150 thereof having been advanced into the subject (e.g., percutaneously advanced, such as transluminally advanced). By way of illustration and not limitation, cannula 150 is shown having been advanced transfemorally, via inferior vena cava 12 of the subject, transseptally into left atrium 14 of the heart, and into left ventricle 16 of the heart. For some applications, and as shown in FIGS. 8A-B, cannula 150 is advanced via an outer sheath 190. FIG. 8A shows guidewire 42 disposed within cannula 150. For example, cannula 150 may have been advanced to the heart over guidewire 42. Alternatively, guidewire 42 may have been advanced through cannula 150 (e.g., using feeder 40) subsequent to the advancement of the cannula. For some applications, it is desirable to subsequently move guidewire 42 with respect to device 180 (e.g., cannula 150 thereof).

Moving guidewire 42 proximally with respect to device 180 is typically not challenging. For example, to withdraw guidewire 42 from the body of the subject, a pulling force is typically applied to the guidewire to pull the guidewire proximally while holding device 180 still. Similarly, to initially advance device 180 over guidewire 42, device 180 is pushed distally while maintaining a pulling force to the guidewire to hold the guidewire still. In contrast, and as described hereinabove, moving guidewire 42 distally with respect to device 180 (e.g., by applying a pushing force to the guidewire) is typically difficult and/or inefficient due to bending of the portion of the guidewire that is between the point of pushing and a point of entry of the guidewire into device 180. Guidewire feeder 40 may be used to initially advance (e.g., push) guidewire 42 through medical device 180, such as in a manner described hereinabove, mutatis mutandis. Furthermore, and as shown in FIGS. 8A-B, feeder 40 may be used to facilitate withdrawal of device 180 from the body of the subject, while keeping guidewire 42 still with respect to the body of the subject, e.g., so as to facilitate the subsequent advancement of another medical device and/or an implant over the same guidewire.

As described hereinabove, FIG. 8A shows cannula 150 of medical device 180 having been advanced into the subject, and guidewire 42 disposed within the cannula. An operating physician withdraws cannula 150 proximally (e.g., by pulling handle 182 proximally) while simultaneously pushing second housing 46 of feeder 40 toward first housing 44, thereby applying a pushing force on guidewire 42, such that the distal end of the guidewire remains stationary with respect to the body of the subject (FIG. 8B). Typically, the distance and/or speed that second housing 46 is pushed toward first housing 44 is relative (although not necessarily identical) to the distance and/or speed that the cannula is withdrawn. Second housing 46 is subsequently moved away from first housing 44, and the process is repeated (e.g., the second housing is oscillated) until a desired degree of withdrawal of cannula 150 (e.g., complete withdrawal from the body of the subject) is achieved. Typically, the operating physician holds medical device 180 (e.g., handle 182 thereof) with one hand, and operates feeder 40 with the other hand (e.g., by holding second housing 46 with the thumb and forefinger).

Guidewire feeder 40 is particularly advantageous in transluminal (e.g., transfemoral) cardiac procedures, for which withdrawal of cannula 150 typically comprises withdrawal over greater than 80 cm (e.g., greater than 1 m), e.g., due to the increased ‘stroke’ length described hereinabove. For some applications, imaging (e.g., fluoroscopy and/or echo) are used to observe the position of cannula 150 and/or guidewire 42 within the body of the subject, such as in real-time so as to facilitate maintenance of the position of the distal end of the guidewire, while retracting cannula 150 from the subject.

Reference is again made to FIGS. 7 and 8A-B. It is to be noted that guidewire feeder 140, described with reference to FIG. 7, may also be used with the techniques described with reference to FIGS. 8A-B.

Reference is again made to FIGS. 1-8. Typically, second housing 46 is shaped to define a handle that is configured (e.g., shaped) to facilitate gripping and operation thereof by the operating physician using only a thumb 122 and forefinger 124 (e.g., as shown in FIG. 6). For some applications, such gripping of second housing 46 by the operating physician is the only point at which the operating physician directly contacts guidewire feeder 40.

Reference is again made to FIGS. 1-8. Although first housing 44 and second housing 46 are shown throughout in the same rotational position around axis a1 with respect to each other, the housings are typically rotatable around axis a1 with respect to each other. For example, while first housing 44 is coupled to medical device 41, second housing 46 is typically freely rotatable around axis a1. For some applications, such free rotation is facilitated by tubular member 48 being freely rotatable within first channel 56. Such free rotation may facilitate effective and/or comfortable use of guidewire feeder 40 by the operating physician. For some applications, this free rotation is partly inhibited while second housing 46 is being pushed toward first housing 44, and guidewire-engaging element 60 is gripping guidewire 42 (e.g., due to the gripping of the guidewire by the guide wire-engaging element).

Reference is again made to FIGS. 1-8. It is to be noted that, although the example of a guidewire (i.e., guidewire 42) is used throughout this application, feeder 40 may also be used to facilitate feeding of other longitudinal (and typically flexible) elements, such as tubular longitudinal elements, e.g., catheters, mutatis mutandis.

Reference is again made to FIGS. 1-8. For some applications, guidewire feeder 40 is integrated with a medical device, such as device 41 or device 180. For example, handle 182 of device 180 may define the first housing of the guidewire feeder, tubular member 48 being slidably coupled to the handle.

It will be understood that, although the terms “first, “second,” etc. may be used in the present application (including the specification and the claims) to describe various elements and/or directions, these terms should not be limiting. These terms are only used to distinguish one element and/or direction from another. Thus, a “first” element described herein could also be termed a “second” element without departing from the teachings of the present disclosure.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method, comprising:
moving a distal end of a percutaneous medical device in a proximal direction away from an anatomical site of a body of a subject; and
simultaneously, maintaining a position, with respect to the anatomical site, of a distal end of a guidewire that is slidable through the medical device by moving, toward a first housing that is coupled to the medical device, a second housing.

2. The method according to claim 1, wherein moving the distal end of the medical device comprises pulling a handle of the medical device in the proximal direction using a first hand, and moving the second housing toward the first housing comprises moving the second housing toward the first housing using a second hand.

3. The method according to claim 1, wherein moving the distal end of the medical device comprises moving the distal end of the medical device at a first speed, and wherein moving the second housing toward the first housing comprises moving the second housing toward the first housing at a second speed that is relative to the first speed.

4. The method according to claim 1, wherein moving the distal end of the medical device comprises moving the distal end of the medical device a first distance, and wherein moving the second housing toward the first housing comprises moving the second housing toward the first housing a second distance that is relative to the first distance.

5. The method according to claim 1, wherein moving the second housing toward the first housing comprises moving the second housing toward the first housing such that a guidewire-engaging element, coupled to the second housing, responsively grips the guidewire.

6. The method according to claim 5, wherein moving the second housing toward the first housing comprises moving the second housing toward the first housing such that the guidewire-engaging element responsively pivots with respect to the second housing.

7. The method according to claim 5, wherein the guidewire-engaging element defines a guidewire-engaging edge, and wherein moving the second housing toward the first housing comprises moving the second housing toward the first housing such that the guidewire-engaging edge presses into the guidewire.

8. The method according to claim 1, further comprising sliding at least the second housing over a proximal end of the guidewire and distally along the guidewire, and coupling at least the second housing to the medical device.

9. The method according to claim 8, wherein sliding at least the second housing distally along the guidewire comprises sliding at least the second housing distally along the guidewire while a guidewire-engaging element, coupled to the second housing, is in a disengaged state thereof, and wherein the method further comprises, subsequently to coupling at least the second housing to the medical device and prior to maintaining the position of the distal end of the guidewire, moving the guidewire-engaging element into an engaged state thereof.

10. The method according to claim 9, wherein moving the guidewire-engaging element into the engaged state comprises moving the guidewire-engaging element into the engaged state using an engagement switch, configured to reversibly move the guidewire-engaging element between the engaged state and the disengaged state.

11. The method according to claim 1, wherein moving the second housing toward the first housing comprises moving the second housing at least 1 cm toward the first housing.

12. The method according to claim 11, wherein moving the second housing toward the first housing comprises moving the second housing at least 3 cm toward the first housing.

13. The method according to claim 12, wherein moving the second housing toward the first housing comprises moving the second housing at least 4 cm toward the first housing.

14. The method according to claim 1, wherein moving the second housing toward the first housing comprises sliding into the first housing, a tubular member that is coupled to the second housing and slidably coupled to the first housing.

15. The method according to claim 14, wherein sliding the tubular member into the first housing comprises sliding a portion of the guidewire that is disposed within the tubular member into the first housing without the portion of the guidewire moving with respect to the tubular member.

16. The method according to claim 15, wherein sliding the tubular member into the first housing comprises sliding, into the first housing, a tubular member that is more rigid than the guidewire and is configured to inhibit bending of the portion of the guidewire.

17. The method according to claim 15, further comprising subsequently sliding the tubular member out of the first housing by moving the second housing away from the first housing, without sliding the guidewire with respect to the first housing.
